(19) United States
(12) Patent Application Publication
(10) Pub. No.: US 2007/0267025 A1
(43) Pub. Date: Nov. 22, 2007

(54) Method and Apparatus for Transnasal Ventilation

(76) Inventors: James R. Lyons, Fairfield, CT (US); Robert G. Russel, Danbury, CT (US)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 10/557,093

(22) PCT Filed: May 20, 2004
(86) PCT No.: PCT/US04/16128

§ 371 Date: Apr. 11, 2007

Related U.S. Application Data
(63) Continuation-in-part of application No. 10/441,557, filed on May 20, 2003.

(51) Int. Cl. A61M 15/08

(52) U.S. Cl. 128/207.18

(57) ABSTRACT

An apparatus and method for delivering oxygen to a nasopharynx and withdrawing exhale gas from the nasopharynx to a carbon dioxide monitor. In one embodiment, the apparatus can comprise one or more tubes and an airway fitting forming an airway. The airway fitting can be configured to engage at least one of the tubes and maintain the one or more tubes within the airway, and to provide an outlet to the atmosphere for the airway. In another embodiment, the tube might be in fluid communication with a junction that can direct oxygen from an oxygen supply through the tube and exhale gas from the tube to the carbon dioxide monitor. Further, the tube might comprise an outer tube and an inner tube.

This application is a continuation-in-part of U.S. patent application Ser. No. 10/441,557, filed on May 20, 2003.

### BACKGROUND

1. Field of the Invention

The present invention relates to the field of respiratory monitoring of carbon dioxide levels and the supplying of oxygen to a patient.

2. Description of the Related Art

It is often desirable or necessary to exchange gas with a subject, such as a medical patient. Using the example of a medical patient, oxygen can be supplied to the patient, and exhale gases such as carbon dioxide can be collected from the patient. When supplying oxygen to the patient, it may be efficient to transfer oxygen to the patient in a stable and controlled location. Likewise, carbon dioxide levels might be monitored more accurately if based on readings taken at a stable and controlled location. Further, when supplying oxygen to or collecting exhale gases from a patient, errors can occur in the setup of the equipment or apparatus. Therefore, gas supply and/or collection equipment or apparatus that can reduce the risk of error can make gas supply and gas collection safer and more reliable.

Thus, there exists a need for a more stable, more efficient, and safer respiratory monitoring and oxygen supply method and apparatus.

### SUMMARY

In an exemplary embodiment, the transnasal ventilation apparatus can both collect carbon dioxide from a patient's nasopharynx and supply oxygen to a patient's nasopharynx through a tube inserted into the nasopharynx. The tube might fluidly communicate with a junction that can direct exhale gas and oxygen through the tube. More particularly, the junction might direct exhale gas from a patient's nasopharynx to a carbon dioxide monitor and/or the junction might direct oxygen from an oxygen supply to a patient's nasopharynx.

In an alternate embodiment, the tube inserted into a patient's nasopharynx might comprise an inner tube and an outer tube. In this embodiment, the inner tube and the outer tube might fluidly communicate with a junction that can direct exhale gas and oxygen through the inner tube and the passageway formed by the inner and outer tubes. More particularly, the junction might direct exhale gas from a patient's nasopharynx to a carbon dioxide monitor and/or the junction might direct oxygen from an oxygen supply to a patient's nasopharynx.

In other embodiments, the transnasal ventilation apparatus can comprise a first tube and a second tube, each in fluid communication with a patient's nasopharynx; and an airway fitting engaging the first tube and the second tube, the airway fitting also in fluid communication with a patient's nasopharynx and with the atmosphere.

In still other embodiments, the transnasal ventilation apparatus can comprise one or more tubes; an airway fitting comprising a walled section forming an airway, the airway fitting being configured to engage at least one of the tubes and maintain the at least one of the tubes within the airway, and wherein the airway fitting is also configured to provide an outlet to the atmosphere for the airway.

In still other embodiments, a airway fitting for use in a transnasal ventilation apparatus can comprise a walled section forming an airway; and one or more members attached to the walled section and configured to engage one or more tubes.

And in still other embodiments, a method can comprise providing a first tube and a second tube, each in fluid communication with a patient's nasopharynx; and providing a airway fitting engaging the first tube and the second tube, the airway fitting also in fluid communication with the patient's nasopharynx and with the atmosphere.

The design of the exemplary embodiments minimize the risk that the apparatus will become dislodged during surgery. In addition, the exemplary embodiments increase safety and control during medical procedures because they maintain oxygen delivery in a more “constant flow” state by supplying constant, passively delivered oxygen to a patient's pharynx. The constant oxygen delivery allows for deeper, more controlled sedation (anesthesia) of the patient. In addition, the exemplary embodiments minimize intrusion on the surgical field of the face.

### BRIEF DESCRIPTION OF THE DRAWINGS

Exemplary embodiments of the present invention are described herein with reference to the drawings, in which:

FIG. 1 is an illustration of an exemplary embodiment;

FIG. 2 is an illustration of a junction shown in FIG. 1;

FIG. 3 is an illustration of another exemplary embodiment;

FIG. 4 is an illustration of another exemplary embodiment;

FIG. 5 is an illustration of another exemplary embodiment;

FIG. 6 is an illustration of another exemplary embodiment;

FIG. 7 is an illustration of several components of an exemplary embodiment;

FIG. 8 depicts an embodiment of an airway;

FIG. 8A depicts a plan view of the airway of FIG. 8;

FIG. 8B depicts a cross-sectional view of the airway of FIG. 8A;

FIG. 8C depicts an elevation view of the airway of FIG. 8;

FIG. 8D depicts a detail of the airway of FIG. 8;

FIG. 9 depicts an embodiment of a flow-through airway fitting;

FIG. 9A depicts an elevation view of the airway fitting of FIG. 9;

FIG. 9B depicts a plan view of the airway fitting of FIG. 9;

FIG. 9C depicts a cross-sectional view of the airway fitting of FIG. 9B; and

FIG. 10 depicts an embodiment of an airway with an alternate flow-through airway fitting embodiment.

### DETAILED DESCRIPTION OF EXEMPLARY EMBODIMENTS

### Example 1

### 1. Overview of Exemplary Embodiments

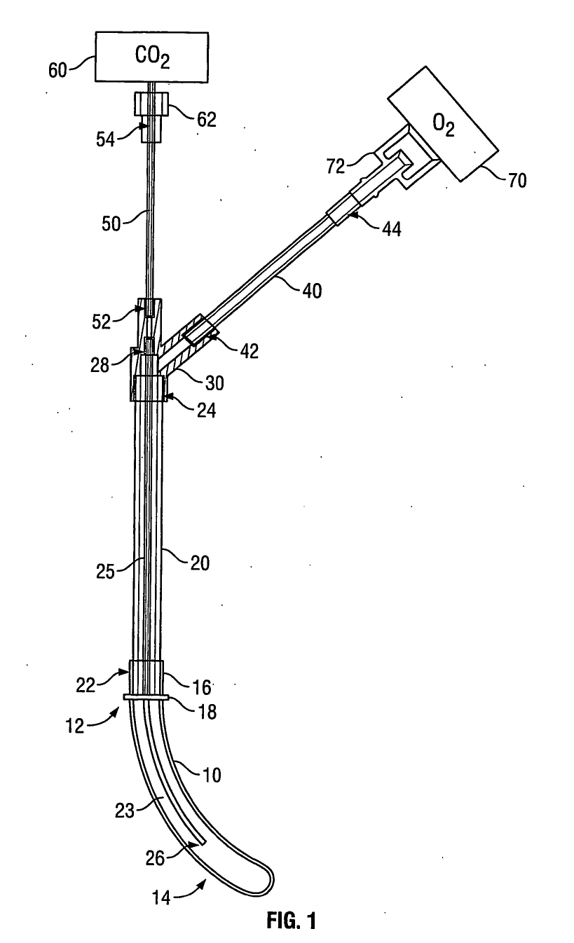

Referring to FIG. 1, in accordance with an exemplary embodiment, a transnasal ventilation apparatus might comprise an insertion guide 10, a first tube 20, a inner tube 25, a junction 30, a second tube 40, and a third tube 50. The first tube 20 might comprise a first end 24 and a second end 22. The inner tube 25 might comprise a first end 26 and a second end 28. The second tube 40 might comprise a first end 42 and a second end 44. And the third tube 50 might comprise a first end 52 and a second end 54. Further, the insertion guide 10, the first tube 20, the inner tube 25, the junction 30, the second tube 40, and the third tube 50 might comprise a single apparatus by, for example, being fused or otherwise bonded together or integral. Other embodiments are possible as well.

Referring to FIG. 1, the insertion guide 10 might comprise a proximal end 12 and a distal end 14. Although it need not be, insertion guide 10 might be tapered. For example, the diameter of the distal end 14 might be larger than the diameter of the proximal end 12. The outside diameters of the proximal end 12 and the distal end 14 may also vary, for example, to accommodate various size nostrils and/or nasal airway passages. The length of the insertion guide 10 may vary as well. In an exemplary embodiment, the distal end 14 of the insertion guide 10 can be inserted into a patient's nasopharynx. In an exemplary embodiment, the insertion guide 10 might be made of a flexible material. For example, the insertion guide 10 might be made of polyvinyl chloride (“PVC”). Other materials, whether flexible or inflexible, are possible as well.

Referring to FIG. 1, in an exemplary embodiment, the proximal end 12 of the insertion guide 10 might comprise a connector 16 and a cuff 18. The connector 16 might receive the first end 22 of the first tube 20. Although not necessary, the connector 16 of the insertion guide 10 might be bonded to the first end 22 of the first tube 20. For example, the connector 16 can be bonded to the first end 22 by an adhesive or through chemical or heat fusing. Other methods of bonding are possible as well. In other embodiments, the connector 16 might be integral with the first end 22. The cuff 18 might contact a patient's nostril and, in addition, might help seal the insertion guide 10 against the patient's nostril.

The first tube 20 might comprise a flexible material, such as PVC. The first tube might also be made of the same material as the insertion guide 10 (which might occur if the insertion guide 10 is integral with or fused to the first tube 20, for instance). Further, the first tube 20 might be made of the same material as the junction 30 (which might occur if the junction 30 is integral with or fused to the first tube 20, for instance). Other 1.5 examples are possible as well.

In an exemplary embodiment, the first tube 20 might comprise a inner tube 25. For example, the inner tube 25 might be inside the first tube 20 such that the outer surface of the inner tube 25 and the inner surface of the first tube 20 can form a passage 23. The passage 23 might, in turn, provide fluid communication between a patient's air passageways and the junction 30.

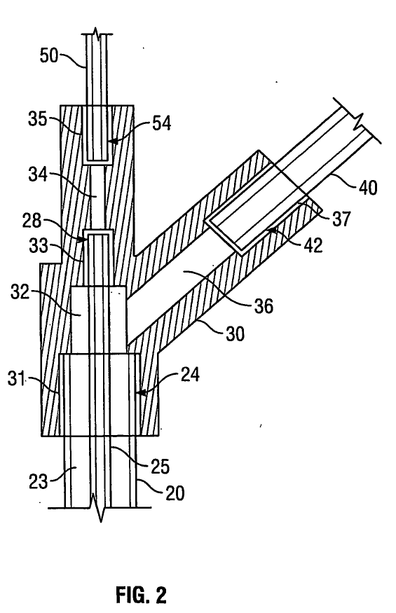

The junction 30 might comprise any type of three-way junction. FIG. 2 depicts an exemplary junction 30 that might comprise seven chambers: a first chamber 31, a second chamber 32, a third chamber 33, a fourth chamber 34, a fifth chamber 35, a sixth chamber 36, and a seventh chamber 37. Other embodiments of junction 30 are possible as well.

In the exemplary embodiments of FIGS. 1 and 2, the first chamber 31 of junction 30 might receive the second end 24 of the first tube 20, and the seventh chamber 37 might receive the first end 42 of the second tube 40. The second chamber 32 and the sixth chamber 36 can then provide fluid communication between the passage 23 and the second tube 40.

Further, the third chamber 33 of junction 30 might receive the second end 28 of the inner tube 25, and the fifth chamber 35 might receive the first end 52 of the third tube 50. The fourth chamber 34 can then provide fluid communication between the inner tube 25 and the third tube 50.

Although not necessary, any combination or all of the first, second, third, or inner tubes 20, 40, 50, and 25 might be bonded to the junction 30. For example, tubes can be bonded to the junction 30 by an adhesive or through chemical or heat fusing. Other methods of bonding are possible as well. In other embodiments, any combination or all of the tubes might be integral with the junction 30.

Other embodiments of the junction 30 and/or the first, second, third, or inner tubes 20, 40, 50, and 25 are possible. For example, portions of the first, second, third, or inner tubes may comprise a single tube. The inner tube 25 and the third tube 50 might comprise a single tube, for instance. In such a case, the third, fourth, and fifth chambers 33, 34, and 35 of junction 30 might comprise a single chamber that can engage the single tube. Other examples are possible as well.

Returning to FIG. 1, the second end 44 of the second tube 40 might be connected to a connector 72. The connector 72 might then connect the second tube 40 to an oxygen supply 70. The second end 54 of the third tube 50 might be connected to a connector 62. The connector 62 might then connect the third tube 50 to a carbon dioxide monitor 60.

The second tube 40 can then fluidly connect the junction 30 to the oxygen supply 70, and the third tube 50 can then fluidly connect the junction 30 to the carbon dioxide monitor 60. The second tube 40 and the third tube 50 might each be made of a flexible material, such as PVC. Other examples are possible as well. For instance, the material of the second tube 40 and the third tube 50 might not be flexible, and the material of any of the first tube 20, the inner tube 25, the second tube 40, or the third tube 50 need not be the same as the material of any other tube. Further, the first, second, third, and inner tubes might also all be made of the same material as the junction 30, which might occur if the first, second, third, or inner tubes are integral with or fused to the junction 30, for instance. The lengths of the first, second, third, and inner tubes might also vary.

### 2. Exemplary Operation

Referring to FIG. 1, in an exemplary embodiment, a user such as an anesthesiologist (or any other medical or non-medical person) might insert the insertion guide 10 into a patient's nasal passage such that the distal end 14 of the insertion guide 10 extends toward the patient's nasopharynx. In such an arrangement, the proximal end 12 of the insertion guide 10 might frictionally engage the patient's nostril. In an exemplary embodiment, the distal end 14 of the insertion guide 10 might extend beyond the second end 26 of the inner tube 25. In another embodiment, the distal end 14 might not extend beyond the second end 26.

The cuff 18 of the insertion guide 10 might provide a seal around a patient's nostril, thereby providing for more efficient oxygen supply and exhale gas withdrawal. Further, as shown in the embodiment of FIG. 1, the insertion guide 10, the first, second, third, and inner tubes 20, 40, 50, and 25, the junction 30, and connectors 62 and 72 might comprise a single apparatus, thereby providing for quicker assembly and easier use. The single apparatus might also provide for safer use because there are fewer parts to assemble, thereby lowering the risk of improper assembly or other errors.

Referring back to the exemplary embodiment of FIG. 1, the second tube 40 might provide for fluid communication between the junction 30 and an oxygen supply 70. The oxygen supply 70, in turn, might apply a low, positive pressure through the second tube 40, the sixth and second chambers 36 and 32 of junction 30, and the passage 23. The third tube 50 might provide for fluid communication between the junction 30 and a carbon dioxide monitor 60. The carbon dioxide monitor 60, in turn, might apply a low, negative pressure through the third tube 50, the fourth chamber 34 of junction 30, and the inner tube 25.

In accordance with an exemplary embodiment, the transnasal ventilation apparatus can provide for a steady state oxygen supply to/carbon dioxide collection from a patient. As the patient inhales, the patient can draw the lightly pressurized oxygen from the oxygen supply 70 through the passage 23 into the patient's nasopharynx. As the patient exhales, the patient can overcome the supply pressure of the oxygen in the passage 23 and can discharge the exhale gases from the patient's nasopharynx into the inner tube 25. The negative pressure applied by the carbon dioxide monitor 60 can, in turn, withdraw the exhale gases to the carbon dioxide monitor 60.

### Example 2

### 1. Overview of Exemplary Embodiments

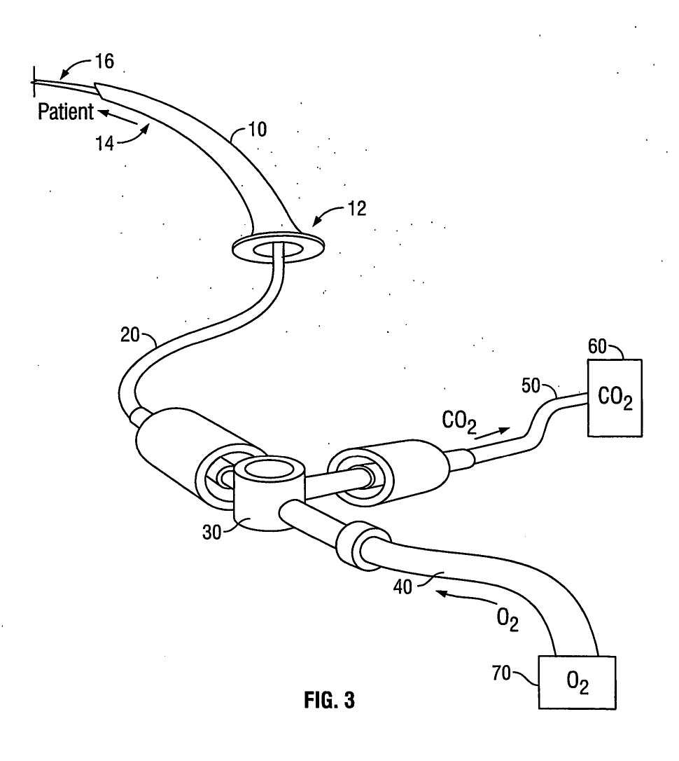

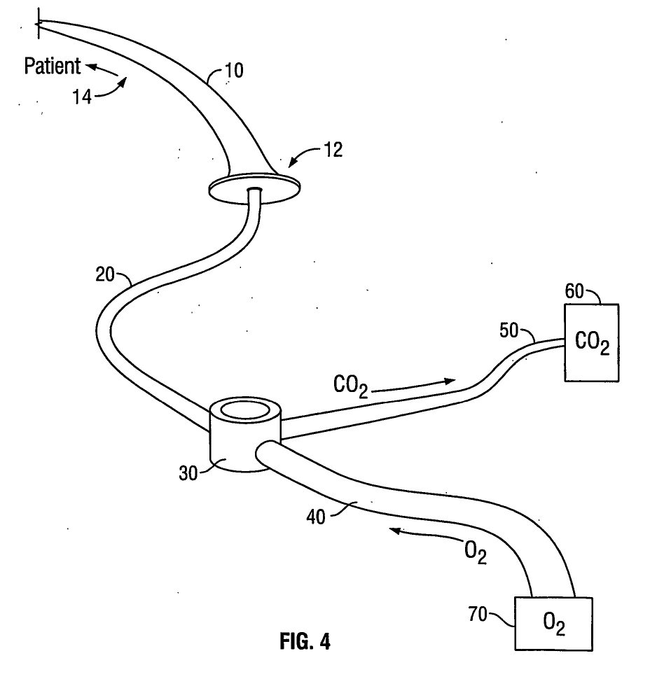

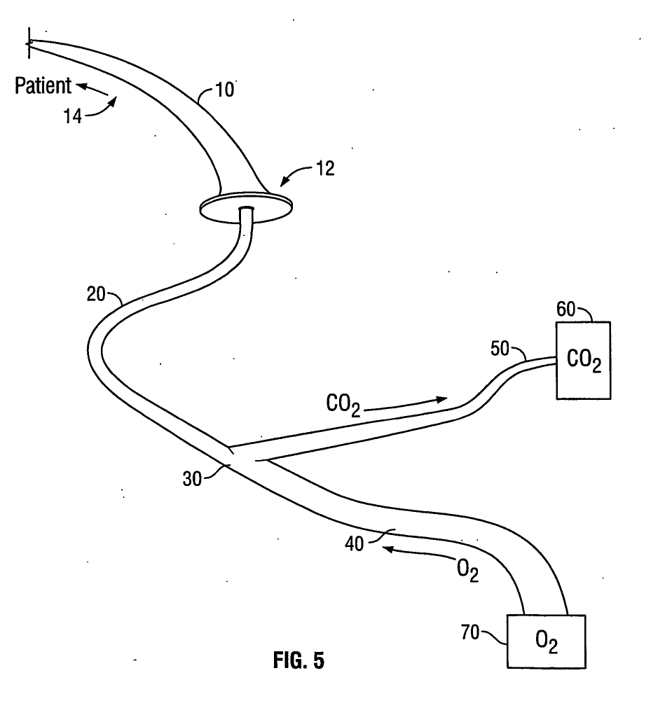

Referring to FIG. 3, in accordance with an exemplary embodiment, a transnasal ventilation apparatus might comprise an insertion guide 10, a first tube 20, a junction 30, a second tube 40, and a third tube 50. Referring to FIG. 4, in accordance with another exemplary embodiment, a transnasal ventilation apparatus might comprise an insertion guide 10, a first tube 20 fixedly attached to the insertion guide 10, a junction 30, a second tube 40, and a third tube 50, the junction 30 being integral with the first, second, and third tubes. FIG. 5 shows an exemplary embodiment similar to the exemplary embodiment of FIG. 4, but with the junction 30 being fused to the first, second, and third tubes. Although not shown, other embodiments are also possible. For instance, in another embodiment, the insertion guide 10 might be fixedly attached to the first tube 20, but the junction 30 might not be integral with or fused to any or all of the first, second, or third tubes. Other examples are possible as well.

Referring to FIGS. 3, 4, and 5, the insertion guide 10 might comprise a proximal end 12 and a distal end 14. Although it need not be, insertion guide 10 might be “bugle” shaped such that the proximal end 12 has a larger circumference than the distal end 14. The outside diameters of the proximal end 12 and the distal end 14 may vary, for example, to accommodate various size nostrils and/or nasal airway passages. In an exemplary embodiment, the outside diameter of the proximal end 12 is 10 mm. In another embodiment, the outside diameter of the proximal end 12 is 8.7 mm. The length of the insertion guide 10 may vary as well.

In an exemplary embodiment, the insertion guide 10 might comprise a cannula Two examples of commercially available cannulae are the Kendall Argyle™ Nasopharyngeal Airway and the Robertazzi™ Nasopharyngeal Airway. Other examples are possible as well. In an exemplary embodiment, the insertion guide 10 might be made of a flexible material. For example, the insertion guide 10 might be made of rubber latex. As another example, the insertion guide 10′ might be made of PVC. Other materials, whether flexible or inflexible, are possible as well.

In an exemplary embodiment, the insertion guide 10 might hold within it a first tube 20. As shown in FIG. 3, for example, the first tube 20 might be slidably inserted into the insertion guide 10. As shown in the embodiments of FIGS. 4 and 5, the first tube 20 might be fixedly attached to the insertion guide 10. For instance, the first tube 20 might be integral with or fused to the insertion guide 10. Other examples are possible as well.

The first tube 20 might comprise a flexible material, such as Silastic™. The first tube might also be made of the same material as the insertion guide 10 (which might occur if the insertion guide 10 is integral with or fused to the first tube 20, for instance). Further, the first tube 20 might be made of the same material as the junction 30 (which might occur if the junction 30 is integral with or fused to the first tube 20, for instance). Other examples are possible as well.

The first tube 20 might, in turn, provide fluid communication between a patient's air passageways and the junction 30. The junction 30 might comprise any type of three-way junction. In one embodiment, the junction 30 might comprise an Airlife™ Tri-Flo® Control Suction Catheter. As shown in the embodiment of FIG. 4, the junction 30 might be integral with the first tube 20, the second tube 40, and the third tube 50. Further, as shown in the embodiment of FIG. 5, the junction 30 might be fused to the first tube 20, the second tube 40, and the third tube 50. Other examples are also possible.

In an exemplary embodiment, the second tube 40 might fluidly connect the junction 30 to an oxygen supply 70, and the third tube 50 might fluidly connect the junction 30 to a carbon dioxide monitor 60. The second tube 40 and the third tube 50 might each be made of a flexible material, such as Silastic™. Other examples are possible as well. For instance, the material of the second tube 40 and the third tube 50 might not be flexible, and the material of any of the first tube 20, the second tube 40, or the third tube 50 need not be the same as the material of any other tube. Further, the first, second, and third tubes might also all be made of the same material as the junction 30, which might occur if the first, second, and third tubes are integral with or fused to the junction 30, for instance. The lengths of the first tube 20, the second tube 40, and the third tube 50 might also vary.

### 2. Exemplary Operation

Referring to FIG. 3, in an exemplary embodiment, a user such as an anesthesiologist (or any other medical or non-medical person) might insert the insertion guide 10 into a patient's nasal passage such that the distal end 14 of the insertion guide 10 extends toward the patient's nasopharynx. The user can then insert a first, open end 16 of the first tube 20 through the insertion guide 10, such that the first end 16 extends toward the patient's nasopharynx. In such an arrangement, the proximal end 12 of the insertion guide 10 might frictionally engage the patient's nostril. The distal end 14 of the insertion guide 10 might frictionally engage the first end 16 of the first tube 20 and thereby hold the first end 16 in place. In an exemplary embodiment, the insertion guide 10 might hold the first end 16 in place beyond the distal end 14. In another embodiment, the first end 16 might not extend beyond the distal end 14. The first end 16 might also be held in place in other ways as well.

As shown in the embodiments of FIGS. 4 and 5, the insertion guide 10 might be fixedly attached to the first tube 20. The insertion guide 10 might then frictionally engage the nostril and thereby be held in place. In the embodiments of FIGS. 4 and 5, the insertion guide 10 and the integral or fused first tube 20 might provide a seal around a patient's nostril, thereby providing for more efficient oxygen supply and exhale gas withdrawal. Further, as shown in the embodiments of FIGS. 4 and 5, the insertion guide 10 and the first tube 20 might comprise a single component, thereby providing for quicker assembly and easier use. The single insertion guide 10/first tube 20 might also provide for safer use because there are fewer parts to assemble, thereby lowering the risk of improper assembly or other errors.

Referring back to the exemplary embodiments of FIGS. 3, 4, and 5, the second tube 40 might provide for fluid communication between the junction 30 and an oxygen supply 70. The oxygen supply 70, in turn, might apply a low, positive pressure through the second tube 40. The third tube 50 might provide for fluid communication between the junction 30 and a carbon dioxide monitor 60. The carbon dioxide monitor 60, in turn, might apply a low, negative pressure through the third tube 50.

In accordance with an exemplary embodiment, the transnasal ventilation apparatus can provide for a steady state oxygen supply to/carbon dioxide collection from a patient. As the patient inhales, the patient can draw the lightly pressurized oxygen from the oxygen supply 70 through the second tube 40 and through the first tube 20 into the patient's nasopharynx. As the patient exhales, the patient can overcome the supply pressure of the oxygen in the first tube 20 and can discharge the exhale gases from the patient's nasopharynx into the first tube 20. The negative pressure applied by the carbon dioxide monitor 60 can, in turn, withdraw the exhale gases to the carbon dioxide monitor 60.

### Example 3

### 1. Overview of Exemplary Embodiments

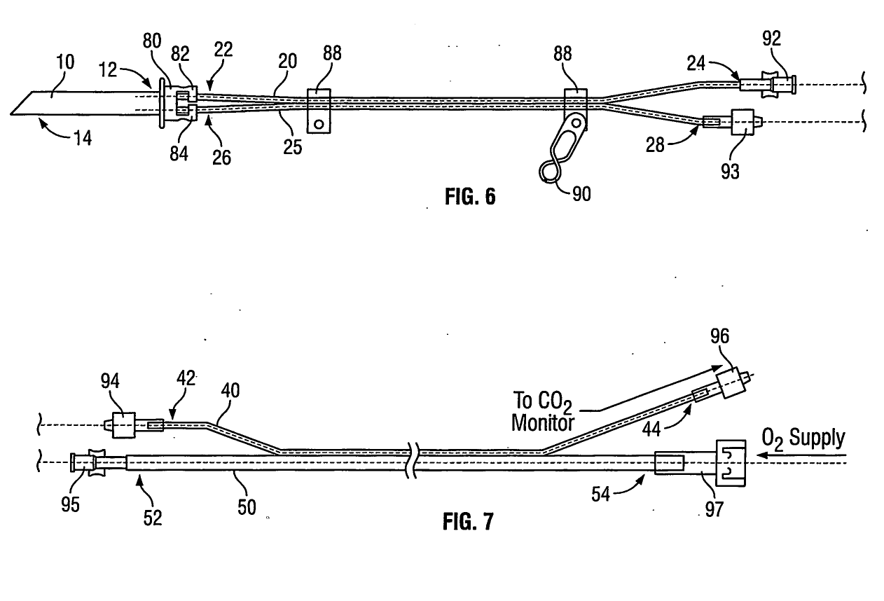

Referring to FIGS. 6 and 7, in accordance with another embodiment, a transnasal ventilation apparatus might comprise an airway, such as an insertion guide 10, a flow-through airway fitting 80, a first tube 20, a second tube 25, a third tube 40, and a fourth tube 50. The first tube 20 might comprise a first end 24 and a second end 22. The second tube 25 might comprise a first end 26 and a second end 28. The third tube 40 might comprise a first end 42 and a second end 44. And the fourth tube 50 might comprise a first end 52 and a second end 54. Although shown as separate components, the insertion guide 10, the flow-through airway fitting 80, the first tube 20, the second tube 25, the third tube 40, and the fourth tube 50 might comprise a single apparatus by, for example, being fused or otherwise bonded together or integral. Other embodiments are possible as well.

As shown in FIG. 6, the first tube 20 and the second tube 25 can be coupled, with one or more cinches 88, for example. (In another embodiment, the tubes can be sold joined together as a pair.) In any case, the tubes can be similar to Datex-Ohmeda No. 73318 tubing, for example. As depicted in FIG. 6, the cinches 88 can prevent the tubing from separating, and can also provide a mount for other devices, such as for a clip 90, for example. The clip 90 can then attach the tubing to the patient, the bed, etc., to make for a neater, safer patient environment.

As shown in FIG. 7, the third tube 40 and the fourth tube 50 can also be coupled. In one embodiment, the tubes can be sold joined together as a pair. (In other embodiments, the tubes can be joined in other ways.) In any case, the tubes can be similar to Datex-Ohmeda No. 73318 tubing, for example. By being joined, the tubes can provide a neater, safer patient environment and can prevent the misconnecting of tubes.

As shown in FIG. 6, the first tube 20 and the second tube 25 can each include one or more fittings on its ends to connect to other components. In one embodiment, the second end 24 of the first tube 20 can comprise a fitting 92, such as a Female Luer Lock, for example. Likewise, the second end 28 of the second tube 25 can comprise a fitting 93, such as a Male Luer Lock, for example. By making the fittings 92 and 93 different (such as by making one a male and one a female fitting, and/or by making the fittings different sizes, for example), the risk of interchanging the tubes is minimized.

As shown in FIG. 7, the third tube 40 and the fourth tube 50 can each also include one or more fittings on its ends to connect to other components. In one embodiment, the first end 42 of the third tube 40 can comprise a fitting 94, such as a Male Luer Lock, for example, and the second end 44 of the third tube 40 can comprise a fitting 96, such as a Male Luer Lock, for example. Likewise, the first end 52 of the fourth tube 50 can comprise a fitting 95, such as a Female Luer Lock, for example, and the second end 54 of the fourth tube 50 can also comprise a fitting 97, which can connect to an oxygen supply or other gas source (or another tube, fitting, component, etc.).

One advantage of using multiple supply and/or exhale tubes is that the length of some of the tubes can be reduced. In one embodiment, the first tube 20 and the second tube 25 can be disposable, and the cost of the disposable portion of the tubing can be reduced by reducing the length of the disposable portion. It can also be easier to pair supply and exhale tubes if multiple supply and/or exhale tubes are used. For instance, by keeping the length of the disposable first tube 20 and the second tube 25 relatively short, the length of the third tube 40 and the fourth tube 50 can be relatively long, and in one embodiment, can be prepackaged as a pair for neater and more convenient routing of the lines from the patient to the oxygen source, carbon dioxide monitor, etc. Other examples are possible as well.

Referring to FIG. 6, the insertion guide 10 might comprise a proximal end 12 and a distal end 14. Although it need not be, insertion guide 10 might be tapered. For example, the diameter of the distal end 14 might be smaller than the diameter of the proximal end 12. The outside diameters of the proximal end 12 and the distal end 14 may also be sized to accommodate various size nostrils and/or nasal airway passages, for example. The insertion guide 10 may be different lengths in different embodiments, but in one embodiment, the insertion guide 10 is long enough to allow the distal end 14 to be inserted into a patient's nasopharynx. In an exemplary embodiment, the insertion guide 10 might be made of a flexible material. For example, the insertion guide 10 might be made of polyvinyl chloride (“PVC”). Other materials, whether flexible or inflexible, are possible as well.

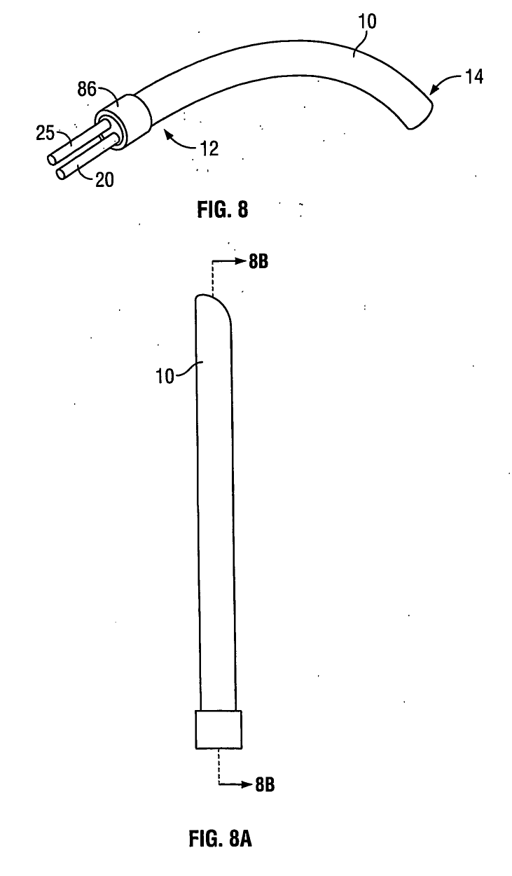

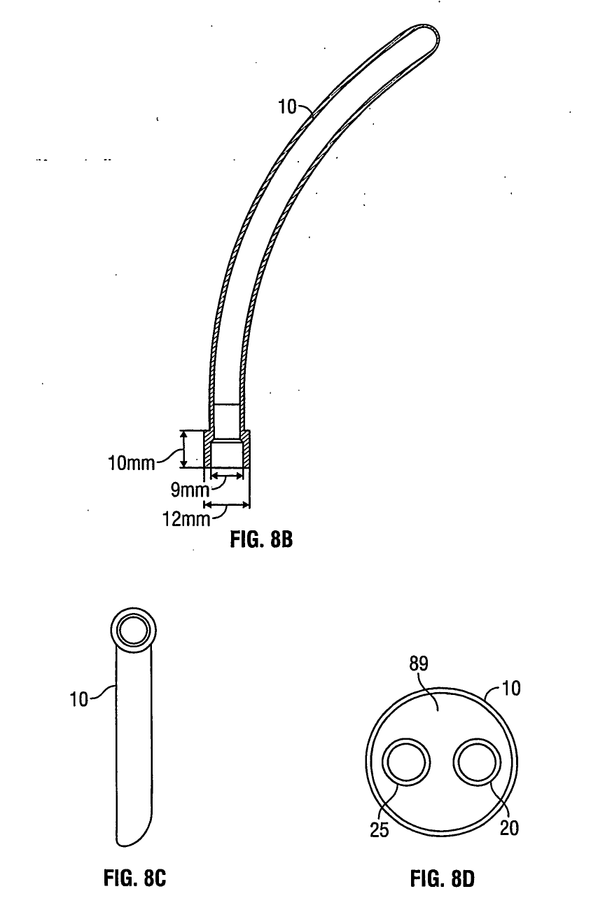

FIG. 8 depicts an exemplary insertion guide 10 around a portion of the first tube 20 and the second tube 25. FIG. 8A depicts a plan view of the insertion guide 10. FIG. 8B depicts a cross-section view of the insertion guide 10, with exemplary dimensions included. FIG. 8C depicts an elevation view of the insertion guide 10. And FIG. 8D depicts a detail of the insertion guide 10 around a portion of the first tube 20 and the second tube 25.

As shown in FIG. 8, the proximal end 12 of the insertion guide 10 can comprise a seat 86. In one embodiment, the seat 86 is 10 mm long, has a 9 mm inside diameter, and has a 12 mm outside diameter.

In one embodiment, the inside diameter at the seat 86 should be large enough to accommodate one or more tubes, such as the first tube 20 and the second tube 25, for example. In one embodiment, the first tube 20 and the second tube 25 each have a 3 mm outside diameter. FIG. 8D depicts a cross-section of the insertion guide 10 at the seat 86, and shows the first tube 20, the second tube 25, and an open space 89. In operation, the open space 89 allows sufficient space for exhaled gas to escape, which, in turn, allows the exhale gas monitor (such as a carbon dioxide monitor) to sample the flow of exhaled gas.

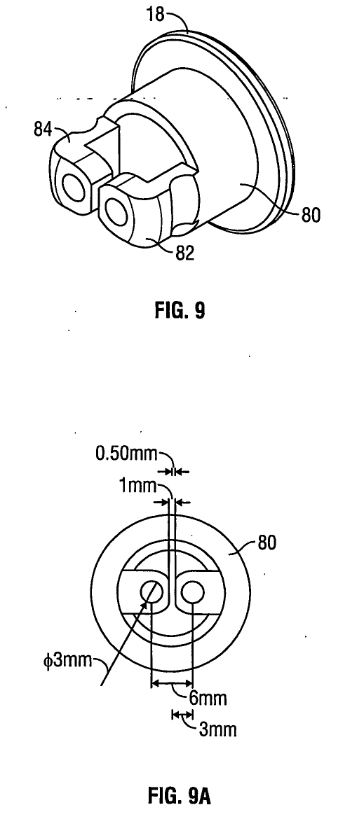

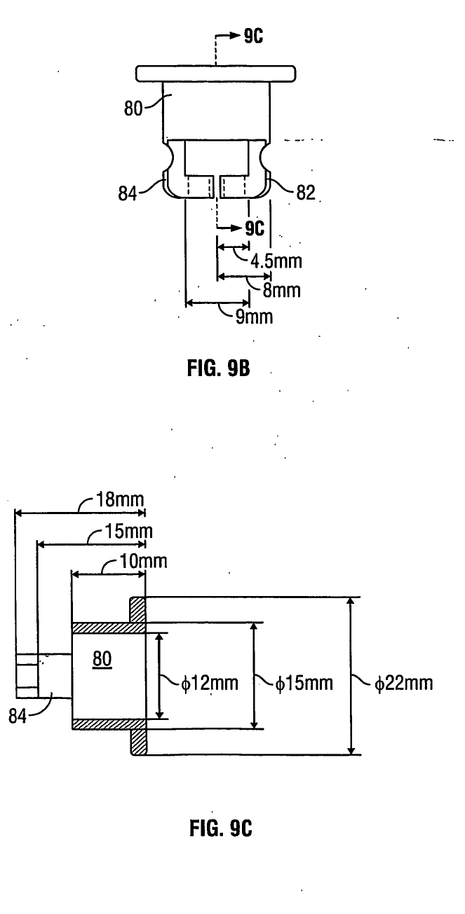

Referring back to FIG. 6, as discussed above, the proximal end 12 of the insertion guide 10 might comprise the flow-through airway fitting 80. In one embodiment, the flow-through airway fitting 80 can engage the seat 86 (shown in FIG. 8) of the insertion guide 10. The airway fitting 80 can also engage a plurality of tubes, such as the first tube 20 and the second tube 25, via one or more arms, such as a first arm 82 and a second arm 84. Each arm might comprise one of any number of mechanisms for engaging one or more tubes, such as an aperture (as shown in FIG. 9) or a clip, for example.

FIG. 9 depicts an exemplary flow-through airway fitting 80. FIG. 9A depicts a plan view of the insertion guide 10. FIG. 9B depicts an elevation view of the insertion guide 10. And FIG. 9C depicts a cross-section view of the insertion guide 10. Some exemplary dimensions are included in these figures, although other examples are possible as well.

As shown in FIG. 9, in one embodiment, the flow-through airway fitting 80 can engage the insertion guide 10 (by being slid onto the proximal end of the insertion guide 10, for example). The airway fitting 80 might also be fixedly attached to the insertion guide 10, such as by chemical or heat bonding or fusing, adhesives, or being integrally formed with the insertion guide 10. Other examples are possible as well.

A feature of one embodiment of the flow-through airway fitting 80 is that it can hold one or more tubes, such as the first tube 20 and the second tube 25, in place in the insertion guide 10, while also providing for and maintaining the opening 89 between the tubes and the inside surface of the insertion guide 10. In one embodiment, the first tube 20 and the second tube 25 can be slid into the openings in the first arm 82 and the second arm 84 of the flow-through airway fitting 80. Other examples are possible as well.

The flow-through airway fitting 80 might also comprise a flange or a cuff 18, which, in one embodiment, can contact a patient's nostril and can help seal the insertion guide 10 against the patient's nostril. The cuff 18 might also facilitate sliding the flow-through airway fitting 80 onto the insertion guide 10.

In one embodiment, the flow-through airway fitting 80 has a 12 mm inside diameter, a 15 mm outside diameter, and is 10 mm long. In one embodiment, the cuff 18 has a 22 mm outside diameter. As shown in FIG. 9C, in one embodiment, the arms 82 and 84 are connected to the non-cuff end of the flow-through airway fitting 80, and extend 5 to 8 mm longitudinally from the non-cuff end. Each arm can also extend transversely into the opening of the flow-through airway fitting 80. FIGS. 9A and 9B show some example dimensions of such a construction. Each arm might also comprise an opening to accommodate a tube, and each opening might have an inside diameter of 3 mm (to accommodate a tube with a 3 mm outside diameter, for example).

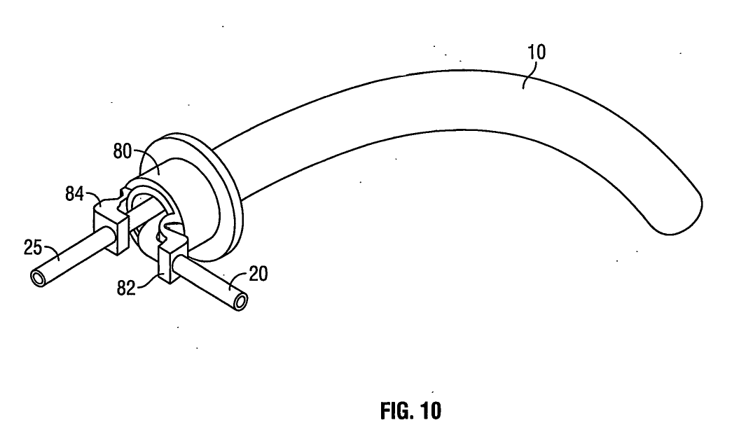

FIG. 10 depicts an alternate embodiment of the flow-through airway fitting 80. In an alternate embodiment, one or more of the arms, such as the first arm 82, of the flow-through airway fitting 80 can be oriented to bend one or more of the tubes (or to accommodate one or more bent tubes), such as the first tube 20, at an (approximately) 90 degree angle. In this way, the bent tube or tubes can be routed in any direction, such as over a patient's ears, allowing access to a patient's face. Other examples are possible as well.

### 2. Exemplary Operation

Referring to FIG. 6, in one embodiment, a user such as an anesthesiologist (or any other medical or non-medical person) might connect exhale and supply tubes, such as the first tube 20 and the second tube 25, to the insertion guide 10. For example, the first tube 20 and the second tube 25 might be threaded through the openings in each of the first arm 82 and the second arm 84 of the flow-through airway fitting 80, as shown in FIG. 6. The first end 22 of the first tube 20 and the first end 26 of the second tube 25 might then each extend into the flow-through airway fitting 80 or the insertion guide 10, and be held in place by the arms of the airway fitting 80.

To deliver oxygen to or exhale gas from a patient's nasopharynx, the user can insert the insertion guide 10 into a patient's nasal passage such that the distal end 14 of the insertion guide 10 extends toward the patient's nasopharynx. The proximal end 12 of the insertion guide 10 might then frictionally engage the patient's nostril, and might provide a seal or partial seal around the patient's nostril.

To place a patient's nasopharynx in fluid communication with a gas source, such as an oxygen source, or with a gas monitor, such as a carbon dioxide monitor, the user might connect the first tube 20 directly to a gas monitor and might connect the second tube directly to a gas source. As shown in FIG. 6, however, the first tube 20 can be in fluid communication with the third tube 40, and the third tube 40 might then directly connect to a gas monitor (or to other tubes, connections, etc., which might fluidly communicate with the gas monitor). Likewise, the second tube 25 can be in fluid communication with the fourth tube 50, and the fourth tube 50 might then directly connect to a gas supply (or to other tubes, connections, etc., which might fluidly communicate with the gas supply). Other examples are possible as well.

Thus, in one embodiment, the second tube 25 and the fourth tube 50 might provide for fluid communication between the patient's nasopharynx and an oxygen supply 70. The oxygen supply 70, in turn, might apply a low, positive pressure through the second tube 25 and the fourth tube 50. Likewise, the first tube 20 and the third tube 40 might provide for fluid communication between the patient's nasopharynx and a carbon dioxide monitor 60. The carbon dioxide monitor 60, in turn, might apply a low, negative pressure through the first tube 20 and the third tube 40.

In accordance with one embodiment, the transnasal ventilation apparatus can provide for a steady state oxygen supply to/carbon dioxide collection from a patient. As the patient inhales, the patient can draw the lightly pressurized oxygen from the oxygen supply through the fourth tube 50 and through the second tube 25 into the patient's nasopharynx. As the patient exhales, the patient can discharge the exhale gases from the patient's nasopharynx into and through the first tube 20 and the third tube 40 to the carbon dioxide monitor, and through the opening 89 in the airway fitting 80 to the atmosphere. The negative pressure applied by the carbon dioxide monitor 60 can, in turn, withdraw the exhale gases to the carbon dioxide monitor 60.

Both supply gas and exhale gas flow to and from the patient's nasopharynx can be enhanced by the opening 89 in the flow-through airway fitting 80. For example, as the patient exhales, some exhaled gas can escape through the opening 89 to the ambient air. The opening 89, and the resultant escaped gas, can be important because some exhale gas monitors need to sample a flow of exhale gas to function properly. The opening 89 can help prevent the exhale gas flow from “dead-ending,” and can encourage and facilitate gas flow to the exhale gas monitor.

### CONCLUSION

Several exemplary embodiments of the present invention have been described above. Those skilled in the art will understand, however, that changes and modifications may be made to these embodiments without departing from the true scope and spirit of the present invention, which is defined by the claims.

We claim:

1. A transnasal ventilation apparatus comprising:
a first supply tube and a first exhale tube, each in fluid communication with a fitting member;
a second supply tube and a second exhale tube, the second supply tube in fluid communication with each of the first supply tube and the fitting member, and the second exhale tube in fluid communication with each of the first exhale tube and the fitting member; and
wherein the fitting member allows delivery of oxygen to a nasopharynx through the first supply tube and allows delivery of exhale gases from the nasopharynx through the first exhale tube.

2. The apparatus of claim 1, in combination with an oxygen source and a carbon dioxide monitor, wherein the second supply tube directs oxygen from the oxygen source to the fitting member and the second exhale tube directs carbon dioxide from the fitting member to the carbon dioxide monitor.

3. The apparatus of claim 1, wherein the fitting member, the first supply tube, the first exhale tube, the second supply tube, and the second exhale tube comprise a single apparatus.

4. The apparatus of claim 1, wherein a portion of the first exhale tube is inside a portion of the first supply tube.

5. The apparatus of claim 1, wherein a portion of the first supply tube is inside a portion of the first exhale tube.

6. The apparatus of claim 1, wherein the first exhale tube and the second exhale tube comprise a single exhale tube.

7. The apparatus of claim 1, wherein the first supply tube and the second supply tube comprise a single supply tube.

8. The apparatus of claim 1, wherein the first supply tube comprises an insertion guide.

9. A method comprising:
delivering oxygen from an oxygen source to a nasopharynx through a first supply tube and delivering exhale gas from the nasopharynx to a carbon dioxide monitor through a first exhale tube, wherein the first supply tube is in fluid communication with a fitting member and a second supply tube, and the first exhale tube is in fluid communication with the fitting member and a second exhale tube.

10. The method of claim 9, wherein the second supply tube directs oxygen from the oxygen source to the fitting member and the second exhale tube directs carbon dioxide from the fitting member to the carbon dioxide monitor.

11. The method of claim 9, wherein at least a portion of the first exhale tube is inside at least a portion of the first supply tube.

12. The method of claim 9, wherein at least a portion of the first supply tube is inside at least a portion of the first exhale tube.

13. The apparatus of claim 9, wherein the first exhale tube and the second exhale tube comprise a single exhale tube.

14. The apparatus of claim 9, wherein the first supply tube and the second supply tube comprise a single supply tube.

15. The apparatus of claim 9, wherein the fitting member, the first supply tube, the first exhale tube, the second supply tube, and the second exhale tube comprise a single apparatus.

16. A transnasal ventilation apparatus comprising:
a first tube in fluid communication with a fitting member;
a second tube and a third tube, each in fluid communication with the first tube and the fitting member; and
wherein the fitting member allows delivery of oxygen to a nasopharynx through the first tube and allows delivery of exhale gases from the nasopharynx through the first tube.

17. The apparatus of claim 16, in combination with an oxygen source and a carbon dioxide monitor, wherein the second tube directs oxygen from the oxygen source to the fitting member and the third tube directs carbon dioxide from the fitting member to the carbon dioxide monitor.

18. The apparatus of claim 16, wherein the fitting member, the first tube, the second tube, and the third tube comprise a single apparatus.

19. The apparatus of claim 16, wherein the first tube comprises an inner tube, and wherein the first tube and the inner tube comprise a passage.

20. The apparatus of claim 19, wherein the passage is in fluid communication with the second tube and the inner tube is in fluid communication with the third tube.

21. The apparatus of claim 20, wherein the inner tube and the third tube comprise a single tube.

22. The apparatus of claim 19, wherein the fitting member, the first tube, the inner tube, the second tube, and the third tube comprise a single apparatus.

23. The apparatus of claim 16, wherein the fitting member, the first tube, the second tube, and the third tube comprise a single apparatus.

24. A method comprising:
delivering oxygen from an oxygen source to a nasopharynx and delivering exhale gas from the nasopharynx to a carbon dioxide monitor through a first tube, wherein the first tube is in fluid communication with a fitting member, a second tube, and a third tube.

25. The method of claim 24, wherein the second tube directs oxygen from the oxygen source to the fitting member and the third tube directs carbon dioxide from the fitting member to the carbon dioxide monitor.

26. The method of claim 24, wherein the first tube comprises an inner tube, and the first tube and the inner tube comprise a passage, wherein the passage is in fluid communication with the second tube and the inner tube is in fluid communication with the third tube.

27. The method of claim 26, wherein the fitting member, the first tube, the inner tube, the second tube, and the third tube comprise a single apparatus.

28. The method of claim 26, wherein the inner tube and the third tube comprise a single tube.

29. A transnasal ventilation apparatus comprising:
a first supply tube in fluid communication with a second supply tube; and
a first exhale tube in fluid communication with a second exhale tube;
wherein the first and second supply tubes allow delivery of oxygen to a nasopharynx and the first and second exhale tubes allow delivery of exhale gases from the nasopharynx.

30. The apparatus of claim 29, wherein at least a portion of the first exhale tube is inside at least a portion of the first supply tube.

31. The apparatus of claim 29, wherein at least a portion of the first supply tube is inside at least a portion of the first exhale tube.

32. The apparatus of claim 29, wherein the first and second supply tubes comprise a single supply tube.

33. The apparatus of claim 32, in combination with an oxygen source, wherein the single supply tube directs oxygen from the oxygen source to the nasopharynx.

34. The apparatus of claim 29, wherein the first and second exhale tubes comprise a single exhale tube.

35. The apparatus of claim 34, in combination with a carbon dioxide monitor, wherein the single exhale tube directs carbon dioxide from the nasopharynx to the carbon dioxide monitor.

36. The apparatus of claim 29, further comprising a fitting member in fluid communication with the first and second supply tubes.

37. The apparatus of claim 36, wherein the first and second exhale tubes comprise a single exhale tube.

38. The apparatus of claim 29, further comprising a fitting member in fluid communication with the first and second exhale tubes.

39. The apparatus of claim 38, wherein the first and second supply tubes comprise a single supply tube.

40. The apparatus of claim 29, further comprising a fitting member in fluid communication with the first and second supply tubes and in fluid communication with the first and second exhale tubes.

41. The apparatus of claim 40, in combination with an oxygen source and a carbon dioxide monitor, wherein the second supply tube directs oxygen from the oxygen source to the fitting member and the second exhale tube directs carbon dioxide from the fitting member to the carbon dioxide monitor.

42. A transnasal ventilation apparatus comprising:
an airway fitting engaging a first tube and a second tube;
wherein the airway fitting allows delivery of oxygen to a nasopharynx through the first tube and allows delivery of exhale gas from the nasopharynx through the second tube.

43. The apparatus of claim 42, wherein the airway fitting comprises an opening that allows some exhale gas to flow to the atmosphere.

44. The apparatus of claim 42, further comprising:
a third tube in fluid communication with the first tube, the third tube also being in fluid communication with an exhale gas monitor.

45. The apparatus of claim 42, further comprising:
a fourth tube in fluid communication with the second tube, the fourth tube also being in fluid communication with an oxygen source.

46. A transnasal ventilation apparatus comprising:
a first tube and a second tube, each in fluid communication with a patient's nasopharynx; and
an airway fitting engaging the first tube and the second tube, the airway fitting also in fluid communication with a patient's nasopharynx and with the atmosphere.

47. The apparatus of claim 46, further comprising:
a third tube in fluid communication with the first tube, the third tube being in fluid communication with an exhale gas monitor.

48. The apparatus of claim 47, further comprising:
a fourth tube in fluid communication with the second tube, the fourth tube being in fluid communication with an oxygen source.

49. A transnasal ventilation apparatus comprising:
one or more tubes;
an airway fitting comprising a walled section forming an airway, the airway fitting being configured to engage at least one of the tubes and maintain the at least one of the tubes within the airway; and
wherein the airway fitting is also configured to provide an outlet to the atmosphere for the airway.

50. The apparatus of claim 49, wherein the one or more tubes comprises a first tube and a second tube.

51. The apparatus of claim 50, further comprising a third tube configured to be placed in fluid communication with the first tube.

52. The apparatus of claim 51, further comprising a fourth tube configured to be placed in fluid communication with the second tube.

53. The apparatus of claim 49, wherein the cross-sectional area of any transverse cross-section of the airway is greater than the cross-sectional area of the transverse cross-section of the at least one of the tubes within the airway at that point.

54. A transnasal ventilation apparatus comprising:
a first tube for delivery of oxygen to a nasopharynx and a second tube for delivery of exhale gases from the nasopharynx.

55. A airway fitting for use in a transnasal ventilation apparatus comprising:
a walled section forming an airway; and
one or more members attached to the walled section and configured to engage one or more tubes.

56. The airway fitting of claim 55, wherein at least one of the members includes an opening.

57. The airway fitting of claim 55, wherein at least a portion of at least one of the members is disposed transversely across the airway.

58. The airway fitting of claim 57, wherein at least one of the members engages one or more tubes.

59. The airway fitting of claim 58, wherein the airway fitting is disposed in a patient's nasopharynx.

60. The airway fitting of claim 55, wherein the airway fitting comprises an insertion guide.

61. The airway fitting of claim 60, wherein the insertion guide is disposed in a patient's nasopharynx.

62. A method comprising:
providing a first tube and a second tube, each in fluid communication with a patient's nasopharynx; and
providing a airway fitting engaging the first tube and the second tube, the airway fitting also in fluid communication with the patient's nasopharynx and with the atmosphere.

63. The method of claim 62, further comprising providing a carbon dioxide monitor in fluid communication with the first tube.

64. The method of claim 63, further comprising providing an oxygen source in fluid communication with the second tube.

65. The method of claim 62, further comprising providing a third tube in fluid communication with the first tube.

66. The method of claim 65, further comprising providing a fourth tube in fluid communication with the second tube.

67. The method of claim 65, further comprising providing a carbon dioxide monitor in fluid communication with the first tube and the third tube.

68. The method of claim 66, further comprising providing an oxygen source in fluid communication with the second tube and the fourth tube.
